(19) United States
(12) Patent Application Publication
(10) Pub. No.: US 2025/0120704 A1
(43) Pub. Date: Apr. 17, 2025

(54) SURGICAL STAPLE CARTRIDGES WITH SLEDS CONFIGURED TO BE COUPLED TO A FIRING DRIVER OF A COMPATIBLE SURGICAL STAPLER

(71) Applicant: Cilag GmbH International, Zug (CH)

(72) Inventors: Frederick E. Shelton, IV, Hillsboro, OH (US); Jason L. Harris, Lebanon, OH (US); Adam D. Hensel, Gahanna, OH (US)

(21) Appl. No.: 18/379,808

(22) Filed: Oct. 13, 2023

(51) Int. Cl. A61B 17/072; A61B 17/00

(52) U.S. Cl. CPC A61B 17/072; A61B 2017/00367; A61B 2017/00477; A61B 2017/07271; A61B 2017/07278; A61B 2017/07285

(57) ABSTRACT

Surgical staple cartridges with sled configurations configured to be coupled to a firing driver of a compatible surgical stapling instrument such that the sled is retractable from a fired position within the staple cartridge to a released position wherein the firing driver decouples from the sled at a location that is distal to an unfired position of the sled.

### BACKGROUND

The present invention relates to surgical instruments and, in various arrangements, to surgical stapling and cutting instruments, end effectors, and staple cartridges for use therewith that are designed to staple and cut tissue.

A variety of different replaceable surgical staple cartridges exist and it is important for each staple cartridge to be compatible with the surgical stapling instrument that it is installed in to prevent instrument malfunction and to ensure that the desired tissue cutting and stapling results are attained. Many surgical staple cartridges include an onboard sled that is configured to be axially driven through the cartridge body by a firing driver of the surgical stapling instrument. The sled is configured to actuate a series of staple drivers that are movably supported within the staple cartridge. The drivers support one or more staples or fasteners thereon and, as the drivers are actuated by the sled, the staples or fasteners are driven through the tissue that is clamped between the cartridge and the anvil of the surgical stapler. The staples or fasteners are formed or closed when they contact the anvil.

In some applications, the firing driver of the surgical stapler is equipped with a knife to sever the clamped tissue as it is being stapled. Because the knife is part of the surgical stapler, the knife is reusable with each new cartridge. However, in other applications, the surgical staple cartridge is equipped with its own knife so that a fresh knife is associated with each replaceable cartridge. In such cartridges, the knife is supported on the sled or is otherwise movably supported within the staple cartridge and is configured to be attached to and/or driven by the firing driver of the surgical stapler during a firing stroke. U.S. Pat. No. 11,234,698, entitled STAPLING SYSTEM COMPRISING A CLAMP LOCKOUT AND A FIRING LOCKOUT discloses various sled/knife assemblies and is hereby incorporated by reference herein in its entirety. In some cartridge arrangements, the knife is non-movably attached to the sled such that it protrudes above a deck surface of the cartridge. In other cartridge arrangements, the knife is movably supported on the sled and is movable between a deployed position in which the knife protrudes above the deck surface and a stored position in which the knife is stored below the deck surface. When using either form of cartridge, however, it is important for the sled/knife assembly to be retractable back to a starting position at all times during the stapling process, should the sled/knife assembly become jammed in an exposed position during the stapling process. In some existing cartridge configurations, however, the sled/knife assembly may inadvertently become detached from the firing beam of the surgical stapler before the sled/knife assembly has been safely returned to a retracted position wherein the knife is not exposed and the cartridge may be safely removed from the stapler. When using such cartridges, it is also important for the cartridge to be compatible with the surgical stapler. It is also important to prevent actuation of such cartridges if the cartridges have been previously fired and inadvertently installed in the compatible surgical stapler.

### SUMMARY

In accordance with the present disclosure, a surgical staple cartridge comprising a cartridge body, a plurality of staple cavities, a staple driver, and a sled is disclosed. The cartridge body defines a proximal end portion and a distal end portion. The cartridge body is configured to be operably seated in a jaw of a surgical stapling instrument. The plurality of staple cavities are arranged longitudinally in the cartridge body. The staple driver is movably supported in each staple cavity. Each staple driver is movable between an unfired position and a fired position. The sled is movably supported in the cartridge body. The sled is movable between an unfired position adjacent the proximal end portion to a fired position adjacent the distal end portion. When the sled is in the unfired position, the sled is configured to be releasably coupled to a firing driver of the surgical stapling instrument when the firing driver is driven distally from a starting position into coupling engagement with the sled. The cartridge body is configured to prevent the sled from decoupling from the firing driver during a firing stroke and a retraction stroke and permit the sled to decouple from the firing driver when the sled is retracted to a released position that is distal to the unfired position.

### LISTING OF THE FIGURES

Various features of the embodiments described herein, together with advantages thereof, may be understood in accordance with the following description taken in conjunction with the accompanying drawings as follows:

FIG. 1 is a partial perspective view of an end effector of a surgical stapling instrument with a staple cartridge installed therein, in accordance with the present disclosure;

FIG. 2 is a partial cross-sectional view of a portion of the staple cartridge and end effector of FIG. 1 with a sled of the staple cartridge in an unfired position and a firing driver of the surgical stapling instrument in a starting position;

FIG. 3 is another partial cross-sectional view of the staple cartridge and end effector of FIG. 2 depicting the firing driver advanced from a starting position in FIG. 2 toward the staple cartridge to engage the sled and defeat a driver lockout of the end effector;

FIG. 4 is another partial cross-sectional view of the staple cartridge and end effector of FIG. 2 depicting the firing driver and the sled retracted proximally as a result of a retraction stroke;

FIG. 5 is another partial cross-sectional view of the staple cartridge and end effector of FIG. 2 depicting the firing driver operably disengaged from the sled during the retraction stroke;

FIG. 6 is a partial cross-sectional perspective view of the staple cartridge and end effector of FIG. 2 depicting a flexible tab, detent, or latch of the staple cartridge holding the sled in the unfired position;

FIG. 7 is another partial cross-sectional perspective view of the staple cartridge and end effector of FIG. 2 wherein the flexible tab, detent, or latch of the staple cartridge is preventing the sled from being retracted proximally beyond a released position that is distal to the unfired position;

FIG. 8 is a perspective view of a staple cartridge comprising a sled including a knife in accordance with the present disclosure with the knife illustrated in an exposed configuration;

FIG. 9 is a perspective view of the sled of the staple cartridge of FIG. 8;

FIG. 10 is a cross-sectional perspective view of the staple cartridge of FIG. 8 depicting the sled in a proximal unfired position and the knife of the sled in a retracted configuration;

FIG. 11 is a cross-sectional perspective view of a proximal portion of the staple cartridge of FIG. 8 depicting the knife of the sled engaging a ledge defined within a longitudinal slot of the staple cartridge;

FIG. 12 is another cross-sectional perspective view of the proximal portion of the staple cartridge of FIG. 8 depicting the knife of the sled in its exposed configuration;

FIG. 13 is a cross-sectional perspective view of the staple cartridge of FIG. 8 depicting the sled in a partially-fired position and the knife of the sled in its exposed configuration;

FIG. 14 is a cross-sectional perspective view of the staple cartridge of FIG. 8 depicting the knife of the sled positioned at a distal end of the ledge of FIG. 11;

FIG. 15 is a cross-sectional perspective view of the staple cartridge of FIG. 8 depicting the sled in a distal fired position and the knife of the sled in its retracted configuration;

FIG. 16 is a cross-sectional perspective view of the staple cartridge of FIG. 8 depicting the sled in its proximal unfired position and the knife of the sled in its retracted configuration;

FIG. 17 is a partial cross-sectional plan view of a staple cartridge and end effector of a surgical stapling instrument with a firing driver of the surgical stapling instrument in a starting position and a sled of the staple cartridge in a proximal unfired position;

FIG. 18 is another partial cross-sectional plan view of the staple cartridge and effector of FIG. 17 after the firing driver has been distally driven into latching engagement with the sled;

FIG. 19 is another partial cross-sectional plan view of the staple cartridge and end effector of FIG. 17 depicting the sled advanced distally from its proximal unfired position by the firing driver;

FIG. 20 is a partial cross-sectional plan view of the staple cartridge and end effector of FIG. 17 depicting the sled in a proximal released position after having been retracted by the firing driver during a retraction stroke;

FIG. 21 is a partial exploded perspective view of the sled and the firing driver of the surgical stapling instrument of FIG. 17;

FIG. 22 is a plan view of the sled of the staple cartridge of FIG. 17 in accordance with the present disclosure; and

FIG. 23 is a partial perspective view of the staple cartridge and end effector of FIG. 17 illustrating a portion of the firing driver in cross section.

### DETAILED DESCRIPTION

Applicant of the present application owns the following U.S. patent applications that were filed on even date herewith and which are each herein incorporated by reference in their respective entireties:
U.S. patent application Ser. No. ______, titled METHOD OF OPERATING A SURGICAL STAPLING INSTRUMENT; Attorney Docket No. END9484USNP2/220491-2M;
U.S. patent application Ser. No. ______, titled SURGICAL STAPLING SYSTEMS WITH ADAPTIVE STAPLE FIRING ALGORITHMS; Attorney Docket No. END9484USNP3/220491-3;
U.S. patent application Ser. No. ______, titled LEARNED TRIGGERS FOR ADAPTIVE CONTROL OF SURGICAL STAPLING SYSTEMS; Attorney Docket No. END9484USNP4/220491-4;
U.S. patent application Ser. No. ______, titled CONTROL CIRCUIT FOR ACTUATING MOTORIZED FUNCTION OF SURGICAL STAPLING INSTRUMENT UTILIZING INERTIAL DRIVE TRAIN PROPERTIES; Attorney Docket No. END9484USNP5/220491-5;
U.S. patent application Ser. No. ______, titled PROPORTIONATE BALANCING OF THE FUNCTION IMPACT MAGNITUDE OF BATTERY OUTPUT TO PEAK MOTOR CURRENT; Attorney Docket No. END9484USNP6/220491-6;
U.S. patent application Ser. No. ______, titled MOTOR OPTIMIZATION BY MINIMIZATION OF PARASITIC LOSSES AND TUNING MOTOR DRIVE CONFIGURATION; Attorney Docket No. END9484USNP7/220491-7;
U.S. patent application Ser. No. ______, titled APPARATUS AND METHOD TO REDUCE PARASITIC LOSSES OF THE ELECTRICAL SYSTEM OF A SURGICAL INSTRUMENT; Attorney Docket No. END9484USNP8/220491-8;
U.S. patent application Ser. No. ______, titled SURGICAL TOOL WITH RELAXED FLEX CIRCUIT ARTICULATION; Attorney Docket No. END9484USNP9/220491-9;
U.S. patent application Ser. No. ______, titled WIRING HARNESS FOR SMART STAPLER WITH MULTI AXIS ARTICULATION; Attorney Docket No. END9484USNP10/220491-10;
U.S. patent application Ser. No. ______, titled SURGICAL SYSTEM WITH WIRELESS ARRAY FOR POWER AND DATA TRANSFER; Attorney Docket No. END9484USNP11/220491-11; and
U.S. patent application Ser. No. ______, titled SURGICAL STAPLE CARTRIDGE COMPRISING REPLACEABLE ELECTRONICS PACKAGE; Attorney Docket No. END9484USNP12/220491-12.

Applicant of the present application owns the following U.S. patent applications that were filed on even date herewith and which are each herein incorporated by reference in their respective entireties:
U.S. patent application Ser. No. ______, titled METHOD OF ASSEMBLING A STAPLE CARTRIDGE; Attorney Docket No. END9484USNP13/220491-13M;
U.S. patent application Ser. No. ______, titled CONTROL SURFACES ON A STAPLE DRIVER OF A SURGICAL STAPLE CARTRIDGE; Attorney Docket No. END9484USNP14/220491-14;
U.S. patent application Ser. No. ______, titled INTEGRAL CARTRIDGE STIFFENING FEATURES TO REDUCE CARTRIDGE DEFLECTION; Attorney Docket No. END9484USNP15/220491-15;
U.S. patent application Ser. No. ______, titled STAPLE CARTRIDGE COMPRISING WALL STRUCTURES TO REDUCE CARTRIDGE DEFLECTION; Attorney Docket No. END9484USNP16/220491-16;
U.S. patent application Ser. No. ______, titled PAN-LESS STAPLE CARTRIDGE ASSEMBLY COMPRISING RETENTION FEATURES FOR HOLDING STAPLE DRIVERS AND SLED; Attorney Docket No. END9484USNP17/220491-17;
U.S. patent application Ser. No. ______, titled STAPLE CARTRIDGE COMPRISING A SLED HAVING A DRIVER LIFT CAM; Attorney Docket No. END9484USNP18/220491-18;
U.S. patent application Ser. No. ______, titled STAPLE CARTRIDGE COMPRISING A COMPOSITE SLED; Attorney Docket No. END9484USNP20/220491-20;
U.S. patent application Ser. No. ______, titled SURGICAL INSTRUMENTS WITH JAW AND FIRING ACTUATOR LOCKOUT ARRANGEMENTS LOCATED PROXIMAL TO A JAW PIVOT LOCATION; Attorney Docket No. END9484USNP21/220491-21;
U.S. patent application Ser. No. ______, titled SURGICAL INSTRUMENTS WITH LATERALLY ENGAGEABLE LOCKING ARRANGEMENTS FOR LOCKING A FIRING ACTUATOR; Attorney Docket No. END9484USNP22/220491-22;
U.S. patent application Ser. No. ______, titled DUAL INDEPENDENT KEYED LOCKING MEMBERS ACTING ON THE SAME DRIVE MEMBER; Attorney Docket No. END9484USNP23/220491-23;
U.S. patent application Ser. No. ______, titled ADJUNCTS FOR USE WITH SURGICAL STAPLING INSTRUMENTS; Attorney Docket No. END9484USNP24/220491-24;
U.S. patent application Ser. No. ______, titled ADJUNCTS FOR USE WITH SURGICAL STAPLING INSTRUMENTS; Attorney Docket No. END9484USNP25/220491-25;
U.S. patent application Ser. No. ______, titled JAW CONTROL SURFACES ON A SURGICAL INSTRUMENT JAW; Attorney Docket No. END9484USNP26/220491-26;
U.S. patent application Ser. No. ______, titled ZONED ALGORITHM ADAPTIVE CHANGES BASED ON CORRELATION OF COOPERATIVE COMPRESSION CONTRIBUTIONS OF TISSUE; Attorney Docket No. END9484USNP27/220491-27;
U.S. patent application Ser. No. ______, titled STAPLE CARTRIDGES COMPRISING TRACE RETENTION FEATURES; Attorney Docket No. END9484USNP29/220491-29; and
U.S. patent application Ser. No. ______, titled STAPLE CARTRIDGES COMPRISING STAPLE RETENTION FEATURES; Attorney Docket No. END9484USNP30/220491-30.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working channel through which the end effector and elongate shaft of a surgical instrument can be advanced.

It may be advantageous for a firing driver, such as an I-beam, of a surgical instrument to selectively couple with a sled of a staple cartridge such that the firing driver can retract the sled to a proximal position after the firing stroke is completed. The sled can comprise a knife thereon. Further, the knife of the sled can be housed in a proximal knife housing prior to being fired and after retraction to reduce the chances of a user coming into contact with the knife of the sled of the staple cartridge. A feature on the distal end of the firing driver can be coupled with a feature on the proximal end of the sled. The firing driver is configured to retract the sled no matter how far distally the sled has been advanced from its proximal unfired position. In other words, the firing driver is capable of retracting the sled/knife from a partially fired position and/or a completely fired position.

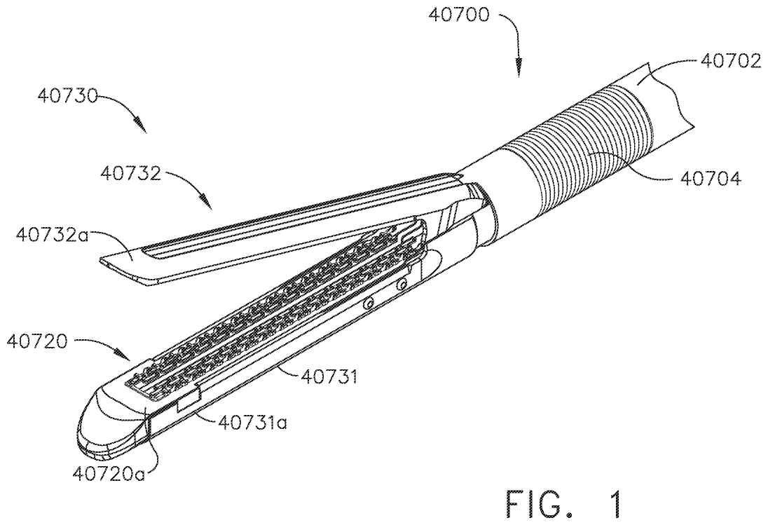

FIGS. 1-6 illustrate a portion of a surgical stapling instrument or surgical stapling system 40700. The surgical stapling instrument 40700 comprises a shaft 40702 and an end effector 40730 extending from the shaft 40702. In the illustrated example, the surgical stapling instrument comprises an articulation joint 40704 positioned intermediate the end effector 40030 and the shaft 40020. The articulation joint 40704 is configured to permit the end effector 40730 to rotate relative to the shaft 40702 about the articulation joint 40704. The end effector 40730 comprises a first jaw 40731 and a second jaw 40732 that is movable relative to the first jaw 40731 between an open position and a closed position to capture the tissue of a patient therebetween. The second jaw 40732 can be movable relative to the first jaw 40731. Referring to FIG. 1, the first jaw 40731 comprises an elongate channel 40731a and the second jaw 40732 comprises an anvil 40732a. The elongate channel 40731a of the first jaw 40731 is configured to receive a replaceable staple cartridge 40720 therein that can be readily removed during a surgical procedure and replaced with a new staple cartridge; however, the first jaw 40731 may alternatively comprise a staple cartridge that is not replaceable. The staple cartridge 40720 comprises a cartridge body 40720a, a sled 40722, a plurality of staple cavities 40750a arranged longitudinally in the cartridge body 40720a, wherein each staple cavity 40750a movably supports a corresponding staple driver 40750 therein, and wherein each staple driver 40750 operably supported one or more staples 40755 thereon.

Further to the above, the surgical stapling instrument 40700 further comprises a firing driver, sled driver or firing actuator 40710 that is movable relative to the end effector 40730 from a proximal position to a distal position during a firing stroke. The firing driver 40710 is configured to axially advance the sled 40722 within the staple cartridge 40720 from a proximal unfired position to a distal fired position during the firing stroke. The firing driver 40710 comprises a proximal portion 40712 and a distal head portion 40714 extending from the proximal portion 40712. In one arrangement, the proximal portion 40712 comprises a laminated beam configured to apply axial drive motions to the distal head portion 40714 yet capable of flexing to accommodate articulation of the end effector 40730. In other arrangements, the proximal portion 40712 may comprise a rod or rods, a beam or beams, an elongated actuator or actuators, etc. and be distally advanced from a starting position during a firing stroke and retracted proximally back to the starting position after the firing stroke by a manually-actuated drive arrangement, a motor-driven drive arrangement, a robotic drive system, a cable-driven system, etc.

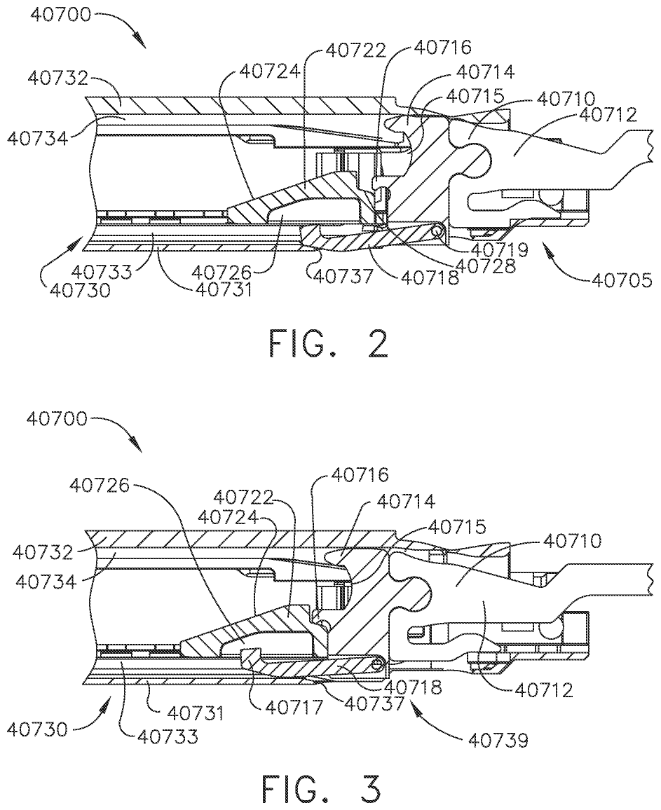

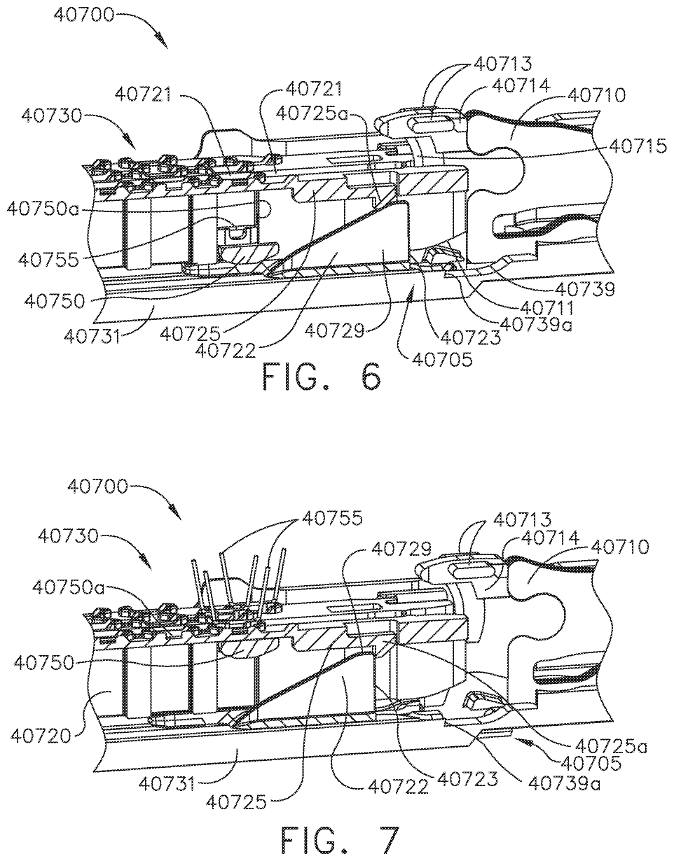

In the illustrated example, the distal head portion 40714 comprises a first lateral cam 40711 (see FIG. 6) that is configured to engage the first jaw 40731 and a second lateral cam 40713 that is configured to engage the second jaw 40732. As shown in FIGS. 2 and 3, the distal head portion 40714 of the firing driver 40710 comprises a knife 40715. However, the sled 40722 may comprise a knife or tissue-cutting feature instead of the distal head portion 40714. In accordance with the present disclosure, the distal head portion 40714 comprises a distal nose 40716 and a latch arm 40718 that is pivotably connected to the distal head portion 40714 via a pin 40719. The latch arm 40718 can be biased downward toward the first jaw 40713 by a biasing member, such as a spring. Other attachment arrangements for pivotably connecting the arm 40718 to the distal head 40714 are contemplated. As can be seen in FIG. 3, a hook portion 40717 is formed at the distal end of the latch arm 40718. As will be discussed in further detail below, the hook portion 40717 is configured to hookingly engage a cavity 40726 in the sled 40722 to latch the sled 40722 to the distal head portion 40714 of the firing driver 40710.

The first jaw 40731 of the end effector 40630 comprises a longitudinal cavity 40733 configured to receive the first cam 40711 of the distal head portion 40714 of the firing driver 40710 during the firing stroke. In addition, the second jaw 40732 comprises a longitudinal cavity 40734 that is configured to receive the second cam 40713 of the distal head portion 40714 of the firing driver 40710 during the firing stroke. The longitudinal cavity 40733 can be further configured to slidably receive the latch arm 40718 and retain the latch arm 40718 in hooking engagement with the sled 40722 throughout the firing stroke and retraction stroke to ensure that the firing driver 40710 can be used to retract the sled at any time during the stapling process. As shown in FIG. 2, a proximal ramp portion 40737 is formed in the first jaw 40731 at the proximal end of the longitudinal cavity 40733 and, as will be discussed in further detail below, serves to move the latch arm 40718 into hooking engagement with the sled 40722 as the firing driver 40710 is distally driven from a starting position.

As can be seen in FIGS. 2, 3, 6, and 7, the sled 40722 comprises a distal ramp 40724 configured to cammingly engage the staple drivers 40750 of the staple cartridge 40720 to eject the staples 40755. FIG. 2 illustrates the first jaw 40731 and the second jaw 40732 in a closed configuration with the sled 40722 of the staple cartridge 40720 in an unfired position and the firing driver 40710 in a proximal position upon initial insertion of the staple cartridge 40720 into the first jaw 40731. The firing driver 40710 is configured to distally advance the sled 40722 through a firing stroke and proximally retract the sled 40722 through a retraction stroke, as discussed in greater detail below.

As was discussed above, when performing various stapling procedures using the surgical stapling instrument 40700, it is important that the staple cartridge 40720 that is installed in the end effector 40730 not only be compatible with the instrument 40700, but it must also be a fresh cartridge that has not been previously fired. If for example, the clinician unwittingly installed a previously fired cartridge into the end effector that was otherwise compatible with the end effector, the tissue may be severed but not stapled or otherwise fastened which could lead to catastrophic results. In accordance with the present disclosure, the surgical stapling instrument 40700 can be equipped with a firing driver lock, generally designated as 40705, which prevents the firing driver 40710 from being distally advanced through the staple cartridge 40720 during an attempted firing stroke. The firing driver lock 40705 comprises a lockout opening 40739 provided in the proximal end of the first jaw 40731. As will be further discussed below, the lockout opening 40739 is sized to receive therein a portion of the distal head portion 40714 of the firing driver 40710 unless a compatible unfired staple cartridge has been operably seated in the first jaw 40731. For example, the first cam 40711 on the distal head portion 40714 can be received in the lockout opening 40739 in the first jaw 40731 unless a compatible, unfired staple cartridge has been seated in the first jaw 40731. When the first cam 40711 of the distal head portion 40714 is received within the lockout opening 40739, the firing driver 40710 is prevent from moving distally should the clinician attempt to initiate the firing stroke. Stated another way, the first cam 40711 will contact a ledge 40739a (FIGS. 6 and F7) formed by the lockout opening 40739 in the first jaw 40731 and thereby prevent distal advancement of the firing driver 40710.

In use, the staple cartridge 40720 is inserted into the first jaw 40731 of the end effector 40730 with the sled 40722 in a proximal unfired position and the firing driver 40710 positioned proximal to the sled 47022. The firing driver 40710 is initially advanced toward the sled 40722 until the distal nose 40716 of the firing driver 40710 engages and rests on a ledge 40728 formed on the sled 40722. The distal nose 40716 of the firing driver 40710 can only rest on the ledge 40728 of the sled 40722 when the sled 40722 is in an unfired position. If the cartridge was previously fired, the sled of the cartridge would not be in the unfired position and could not engage the distal nose 40718 of the firing driver 40710. When the distal nose 40716 is received on the ledge 40728 on the sled 40722, the firing driver 40710 and, more particularly, the distal head portion 40714 of the firing driver 40710, is prevented from being biased into a lockout opening 40739 in the first jaw 40731. The distal head portion 70714 of the firing driver 40710 may be biased toward the lockout opening 40739 in the first jaw 40731 of the end effector 40730 by a biasing member such as a spring, for example. As such, if the firing driver 40710 is distally advanced without the staple cartridge 40720 present, or with the staple cartridge 40720 present and the sled 40722 is not in the proximal unfired position, the distal head portion 40714 of the firing driver 40710 will be biased into the lockout opening 40739 by the biasing member and, thus, the firing driver 40710 will be prevented from moving distally during an attempted firing stroke.

Further to the above, when the firing driver 40710 is advanced from the position in FIG. 2 to the position in FIG. 3, the latch arm 40718 of the firing driver 40710 engages the proximal ramp portion 40737 of the longitudinal cavity 40733 and is driven toward the sled 40722 such that the hook portion 40717 of the latch arm 40718 enters the cavity 40726 of the sled 40722.

The firing driver 40710 is configured to abut and advance the sled 40722 from the position in FIG. 2 to a distal fired position during the firing stroke to eject the staples from the staple cartridge 40720. Further, the hook 40717 of the distal head portion 40714 remains positioned within the cavity 40726 during the firing stroke of the firing driver 40710 due to the interaction between the latch arm 40718 and the longitudinal cavity 40733.

After the firing stroke is completed, or when a partial firing stroke is performed, the firing driver 40710 is configured to retract the sled 40722 proximally to the released position illustrated in FIG. 6 during a retraction stroke. Specifically, the sled 40722 is pulled proximally by the firing driver 40710 during the retraction stroke due to the engagement of the distal hook 40717 of the arm 40718 of the firing driver 40610 with a proximal wall 40726a of the cavity 40726 of the sled 40722. As the firing driver 40710 retracts further proximally from the position in FIG. 5 to its original starting position, the latch arm 40718 pivots away from the sled 40722 to disengage the firing driver 40710 from the sled 40722. As discussed above, the arm 40718 of the firing driver 40710 is biased toward the first jaw 40731 by a biasing member such as a spring or the like. As such, when the firing driver 40710 is retracted, the arm 40718 is biased toward the proximal ramp portion 40737 of the first jaw 40731 and disengaged from the cavity 40726 of the sled 40722. In other words, the first jaw 40731 provides room for the arm 40718 to be biased out of the cavity 40726 of the sled 40722 when the firing driver 40710 is retracted back to its starting position. Further, the spent, or at least partially spent, staple cartridge 40720 can then be removed from the first jaw 40731.

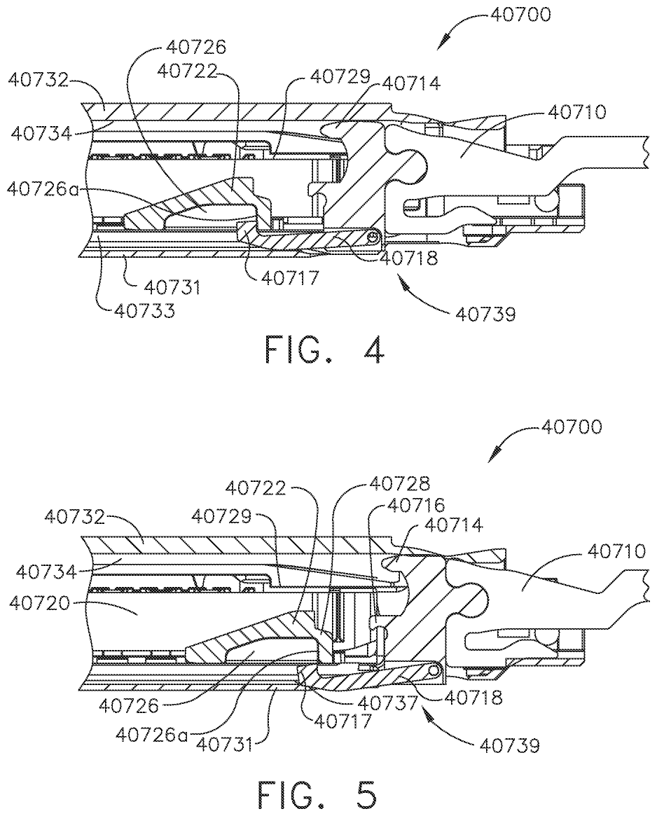

As discussed above, as the firing driver 40710 is retracted back to its starting position, the distal head portion 40714 of the firing driver 40710 disengages from the sled 40722 as depicted in FIGS. 4 and 5. As can be seen in FIG. 5, when the firing driver 40710 is retracted into its original starting position, the sled 40722 has been proximally moved into the released position that is distal to its original proximal unfired position (i.e., the position of the sled 40722 in FIGS. 2 and 3). When the firing driver 40710 is retracted, the first cam 40711 of the firing driver 40710 is biased into the lockout opening 40739 as the distal nose 40716 no longer rests on the ledge 40728 of the sled 40722 to prevent the first cam 40711 from entering the lockout opening 40739. Further, when the distal head portion 40714 of the firing driver 40710 enters the lockout opening 40739, the first cam 40711 is viewable to a user of the surgical stapling instrument 40700 from the bottom of the elongate channel 40731a and therefore indicates to the user that the surgical attachment 40700 is locked out (i.e., the firing driver 40710 is prevented from performing a firing stroke). After the firing driver 40710 is retracted and the latch arm 40718 is disengaged from the sled 40722, the staple cartridge 40720 can be removed from the end effector 40730 and replaced with another staple cartridge 40720 with another sled 40722 in the proximal unfired position. The new unspent staple cartridge can be inserted into the end effector 40730 such that the sled 40722, in its proximal unfired position defats the firing drive lock 40705 so the firing driver 40710 can perform another firing and retraction stroke.

If the spent staple cartridge 40720 with the sled 40722 in the position of FIG. 5 is removed from the end effector 40730 and placed in another surgical stapling instrument identical to the surgical stapling instrument 40700, the firing driver 40710 will not be able to perform a firing stroke. Specifically, when the sled 40722 is in the position of FIG. 5, the distal nose 40716 of the firing driver 40710 will not be able to engage and rest on the ledge 40728 of the sled 40722 when the firing driver 40710 is distally advanced and, thus, the first cam 40711 of the firing driver 40710 will be biased into the lockout opening 40739 preventing the firing driver 40710 from performing the firing stroke through the spent, or at least partially spent, staple cartridge 40720. Moreover, the first cam 40711 positioned in the lockout opening 40739 is viewable to a user of the attachment 40700 and indicates that the firing driver 40710 is locked out and prevented from performing the firing stroke.

Further to the above, the latch arm 40718 of the firing driver 40710 may comprise a unique keyed geometry that can only be received by a complementary keyed relief feature (cavity) in the sled 40722. As such, if a non-compatible staple cartridge is inserted into the end effector 40730, the arm latch 40718 of the firing driver 40710 will not be able to properly engage with the sled of the non-compatible staple cartridge. In such instances, the latch arm 40718 would be prevented from advancing up the proximal ramp portion 40737 and thereby prevent the distal advancement of the firing driver 40710.

As can also be seen in FIGS. 6 and 7, the staple cartridge 40720 further comprises a flexible tab 40725 that is configured to retain the sled 40722 in the proximal unfired position during shipping and during installation into the end effector 40730. FIG. 6 illustrates the position of the sled 40722 in an unfired state. This is the state in which the staple cartridge 40720 is shipped and stored prior to use. As can be seen in FIG. 6, the flexible tab 40725 frictionally engages a corresponding rail 40729 of the sled 40722 and serves to prevent the sled from moving out of that unfired position prior to use. The staple cartridge 40720 may comprise another flexible tab 40725 on the other side of a longitudinal slot 40721 of the staple cartridge 40720 that is configured to engage another rail 40729 of the sled 40722. In any event, the flexible tab, or tabs 40725, are biased toward the first jaw 40731 to engage the sled 40722 to prevent the sled 40722 from inadvertently moving prior to firing the staple cartridge 40720.

In use, as the firing driver 40710 engages the sled 40722 during the firing stroke, the flexible tab 40725 rides up the rail 40729 until the sled 40722 moves distally out of the unfired position and the flexible tab 40725 completely disengages the rail 40729. After the firing stroke has been completed, the sled 40722 is retracted proximally by the firing driver 40710 until the rails 40729 engage the flexible tab 40725 as shown in FIG. 7. As such, when the sled 40722 is retracted during the retraction stroke, the flexible tab 40725 prevents the sled 40722 from returning to its original proximal unfired position (i.e., the position of the sled 40722 in FIG. 6). Specifically, a hook portion 40725a of the flexible tab 40725 engages a proximal face 40723 of the sled 40722 to prevent further proximal retraction of the sled 40722. This position may coincide with the released positioned discussed above, for example. As was further discussed above, if the sled 40722 is not in its proximal unfired position, the firing driver lock 40705 will not be defeated and the firing driver 40710 will be unable to perform a firing stroke.

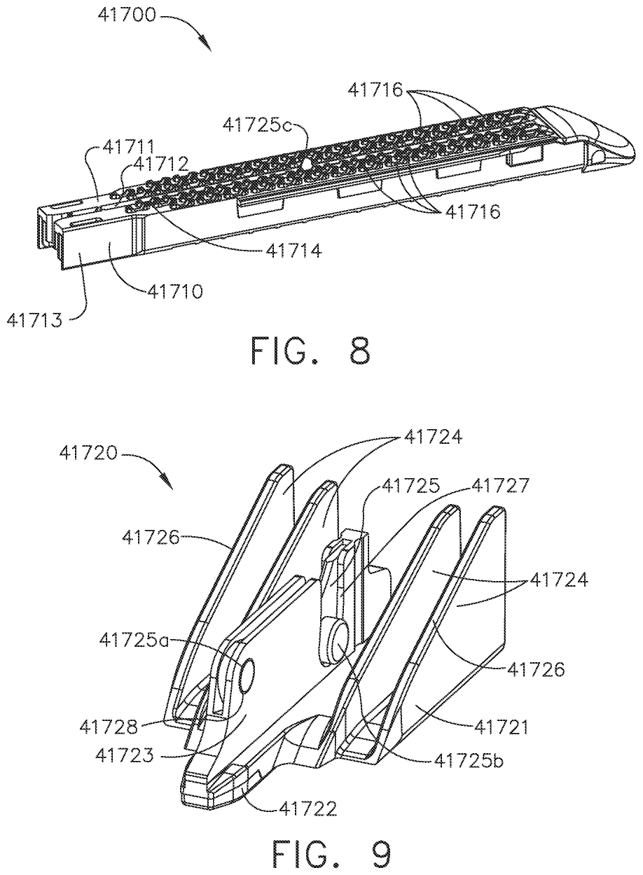

FIGS. 8-16 illustrate a staple cartridge 41700 that comprises a cartridge body 41710 and a sled 41720 that is movable within the cartridge body 41710. The cartridge body 41710 comprises a longitudinal slot 41712, a deck 41714, and a plurality of longitudinal rows of staple cavities 41716 defined in the deck 41714. The longitudinal slot 41712 divides the staple cartridge 41700 into a first side 41711 and a second side 41713 opposite the first side. The first side 41711 and the second side 41713 are connected at the distal end of the cartridge body 41710. Three rows of staple cavities 41716 are positioned on the first side 41711 of a longitudinal slot 41712 and three rows of staple cavities 41716 are positioned on a second side of the longitudinal slot 41712. However, other staple cavity arrangements are envisioned. The staple cartridge 41700 further comprise a plurality of staple drivers movably positioned within the staple cavities 41716. The staple drivers support staples thereon for ejection from the staple cartridge 41700.

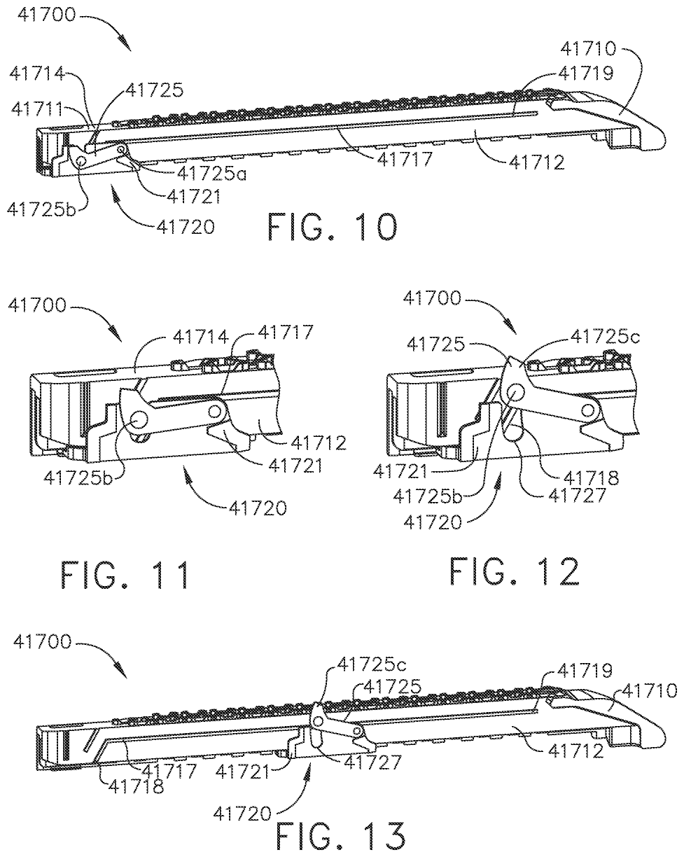

As can be seen in FIG. 10, the cartridge body 41710 comprises a ledge 41717 extending laterally into the longitudinal slot 41712 from the first side 41711 of the cartridge body 41710. The cartridge body 41710 may comprise another ledge extending into the longitudinal slot 41712 from the second side 41713 of the cartridge body 41710 in line with the ledge 41717. In any event, the ledge 41717 extends longitudinally within the longitudinal slot 41712 and comprises a proximal ramp 41718 and a distal end 41719 as depicted in FIG. 13.

Referring now to FIG. 9, the sled 41720 comprises a wedge portion 41721 and a knife 41725 that is rotatable relative to the wedge portion 41721. The wedge portion 41721 comprises a base 41722, a central nose portion 41723 extending upward from the base 41722, and a plurality of rails 41724 extending upward from the base 41722 on either side of the nose portion 41723. Two rails 41724 are positioned on one side of the base 41722 and two rails 41724 are positioned on the opposite side of the base 41722. Each of the rails 41724 comprises a distal ramp 41726 configured to cammingly engage the staple drivers in the cartridge body 41710 to eject the staples from the staple cavities 41716 when the sled 41720 is moved from a proximal unfired position (FIG. 10) to a distal fired position (FIG. 15) during a firing stroke. Further, the central nose portion 41723 is configured to move within the longitudinal slot 41712 of the cartridge body 41710 during the firing stroke.

As can be seen in FIG. 9, a proximal slot 41727 and a distal through hole 41728 is provided in the nose portion 41723. The knife 41725 is pivotably connected to the wedge portion 41721 by a distal pin 41725a that is received within the distal through hole 41728 in the nose portion 41723. As can be further seen in FIG. 9, the knife portion 41725 comprises a proximal pin 41725b that is movably received within the proximal slot 41727 in the nose portion 41723. The proximal pin 41725b is configured to interact with the ledge portion 41717 in the cartridge boy 41710 when the sled 41720 moves therethrough.

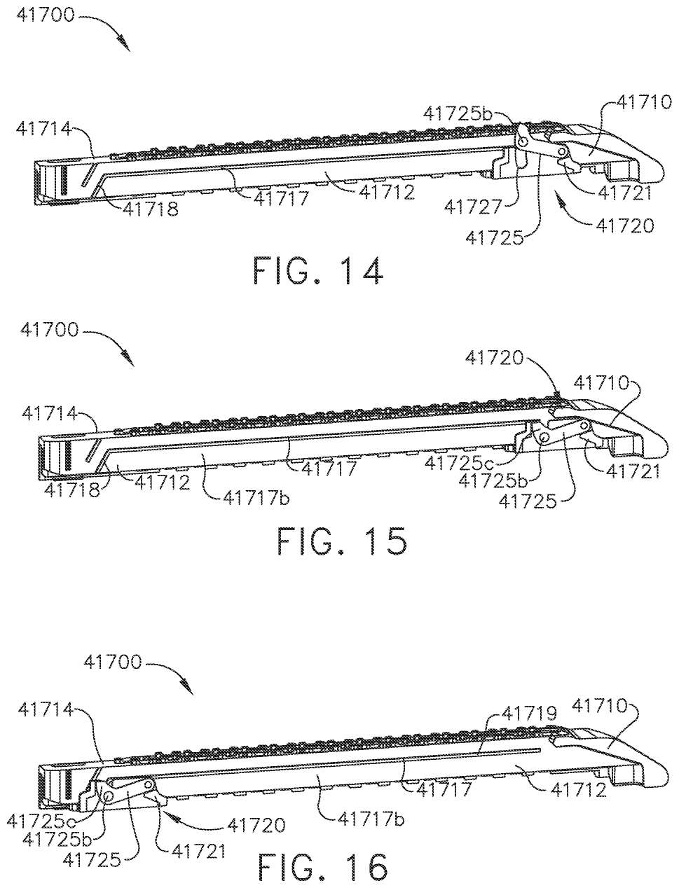

When the staple cartridge 41700 is initially inserted into an end effector of a surgical stapling instrument, the sled 41720 is in a proximal unfired position illustrated in FIG. 10. Specifically, the wedge portion 41721 of the sled 41720 is in a proximal unfired position and the knife 41725 is in a retracted position. When the sled 41720 is advanced from the position in FIG. 10 to the position in FIG. 11, the proximal pin 41725b engages the proximal ramp 41718 of the ledge 41717 in the cartridge body to lift the knife 41725 within the proximal slot 41727 of the wedge portion 41721 toward the deck 41714. Further distal advancement of the sled 41720 from the position in FIG. 11 to the position in FIG. 12, lifts the knife 41725 into a position wherein a cutting portion of the knife protrudes out of the longitudinal slot 41712. For example, when the knife 41725 is in the exposed configuration depicted in FIG. 12, a knife edge 41725c of the knife 41725 is at least partially positioned above the deck 41714 of the cartridge body 41710. Further, the proximal pin 41725b is configured to ride upon the ledge 41717 in the longitudinal slot 41712 to maintain the knife 41725 in the exposed configuration as the sled 41720 is further distally advanced from the position in FIG. 12 to the position in FIG. 14. When the sled 41720 moves from the positon in FIG. 14 to a distal fired position illustrated in FIG. 15, the proximal pin 41725b falls from the distal end 41719 of the ledge 41717 and into the proximal slot 41727 of the central nose portion 41723. As such, the knife 41275 moves from the exposed configuration in FIG. 14 into the retracted configuration illustrated in FIG. 15. The sled 41720 may be permitted to remain in the distal fired position within the staple cartridge 41700 (FIG. 15) and is not retracted back to the proximal end of the staple cartridge. The staple cartridge can then be removed from the end effector with the knife 41725 in a retracted, or non-exposed, configuration and, thus, the possibility of a user coming into contact with the knife 41549 while handling the spent staple cartridge 41700 is reduced.

In accordance with the present disclosure, the sled 41720 may be configured in the various manners discussed herein to enable the sled 41720 to otherwise be retracted from the distal fired position (FIG. 15) to a proximal position illustrated in FIG. 16. The proximal pin 41725b may be maintained in the proximal slot 41727 of the sled 41720 on an underside 41717b of the ledge 41717 during retraction. The ledge 41717 prevents the knife 41725 from becoming exposed above the deck 41714 of the cartridge body 41710 as the sled 41720 is retracted and when the sled is in the proximal position illustrated in FIG. 16. As such, the staple cartridge 41700 can be removed from the end effector with the knife 41725 in a retracted, or non-exposed, configuration and the sled 41720 in the proximal position illustrated in FIG. 16. In addition, in the event the sled 41720 is only partially distally advanced during the firing stroke, such as to the position illustrated in FIG. 13, the firing driver 40710 can be employed to retract the sled 41720 proximally into the position illustrated in FIG. 10 thereby enabling the partially fired staple cartridge 41700 to be removed from the end effector with the knife 41725 in a retracted, or non-exposed position.

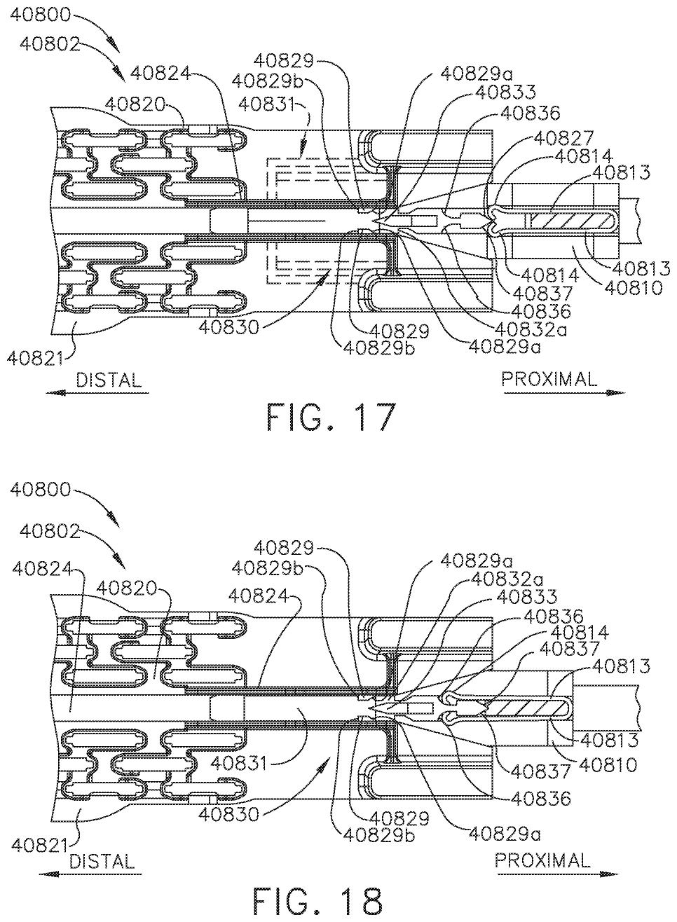

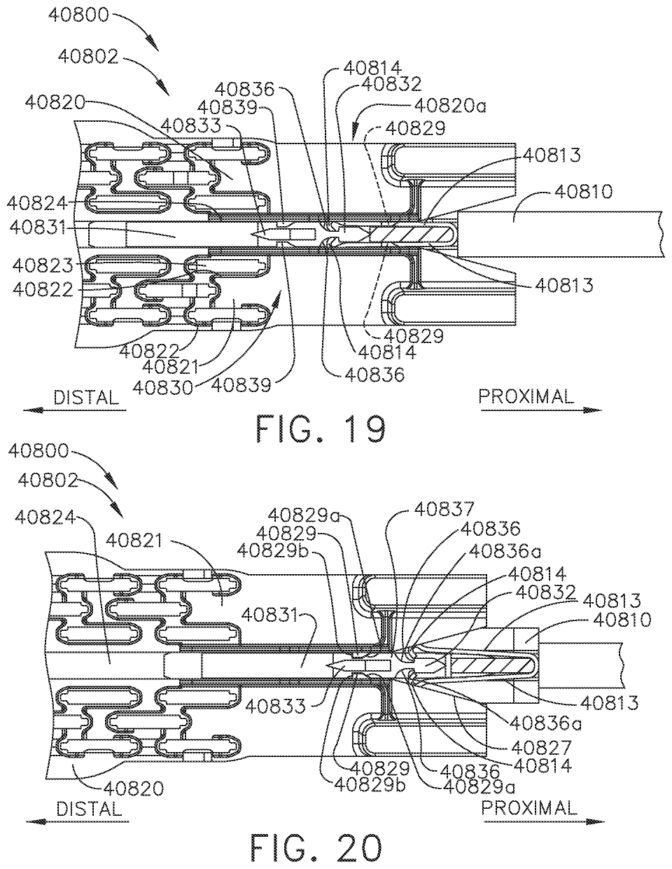

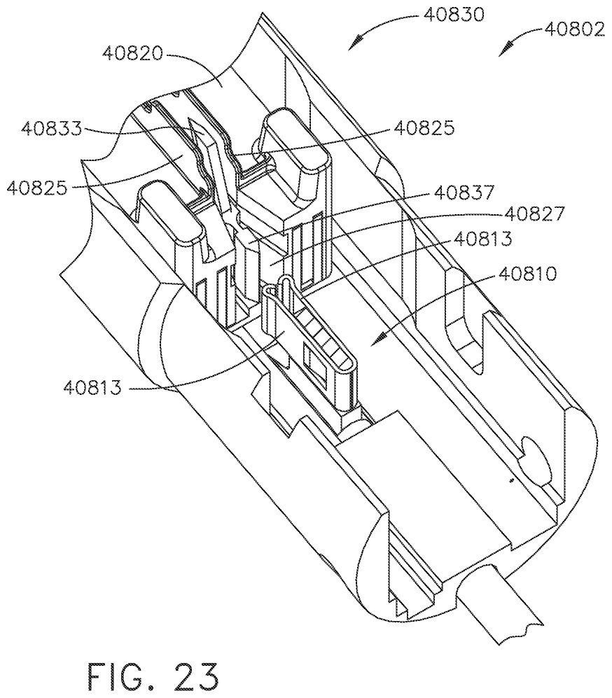

FIGS. 17-23 illustrate a portion of a surgical system 40800 comprising an end effector 40802 configured to receive a staple cartridge 40820. The surgical system 40800 further comprises a firing driver, sled driver or firing actuator 40810 that is movable relative to the end effector 40802. The end effector 40802 comprises a first jaw configured to receive the staple cartridge 40820 and a second jaw movable relative to the first jaw to capture tissue between the first jaw and the second jaw similar to the end effector depicted in FIG. 1, for example. The staple cartridge 40820 may be similar to the staple cartridge 40720 except for the differences noted herein. For example, in the illustrated arrangement, the staple cartridge 40820 comprises a cartridge body 408020a that defines a deck surface 40821. A plurality of staple cavities 40822 are defined in the deck surface 40821. A longitudinal slot 40824 is defined in the deck surface 40821 and divides the staple cartridge 40820 into a first side and a second side opposite the first side. As can be seen in FIG. 23, a proximal portion of the longitudinal slot 40824 comprises a V-cutout portion 40827 that opens through the proximal end of the staple cartridge 40820. As can be seen in FIGS. 17 and 18, a cartridge slot protrusion 40829 protrudes inwardly from each side of the longitudinal slot 40824. Each cartridge slot protrusion 40829 comprises a proximal face 40829a and a distal face 40829b. The proximal faces 40829a are ramped, or tapered, laterally and each of the distal faces 40829b are laterally flat, or substantially flat.

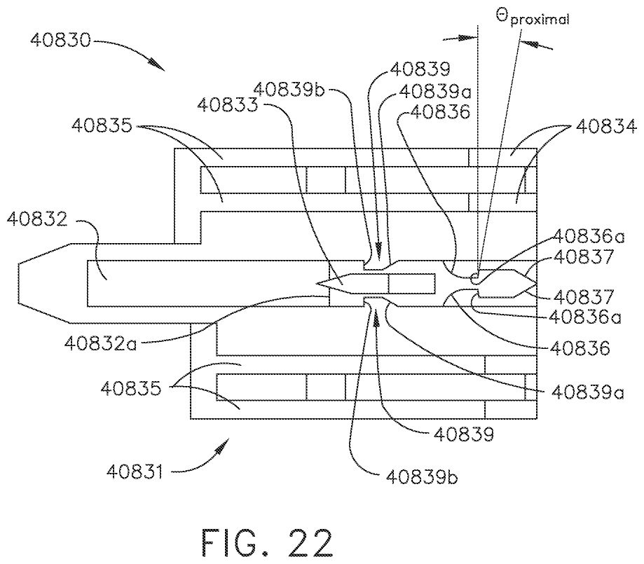

Turning to FIG. 22, the staple cartridge 40820 includes a sled 40830 that comprises a wedge portion 40831 and a nose portion 40832. The wedge portion 40831 comprises a plurality of rails 40834. Each of the rails 40834 comprises a distal ramp 40835 that is configured to cammingly engage staple drivers 40823 (see FIG. 18) to eject staples or fasteners supported on the staple drivers 40823 from the staple cartridge 40820. The nose portion 40832 of the sled 40830 comprises a knife 40833 that is configured to extend above the deck surface 40821. The knife 40833 is configured to incise patient tissue that is captured between the first jaw and the second jaw of the end effector 40802. Further, when the sled 40830 is in a proximal unfired position, the knife 40833 is configured to be stored within a knife housing 40825 formed in the cartridge body. The knife housing 40825 extends above the deck surface 40821 as depicted in FIG. 23.

As can be seen in FIG. 22, the nose portion 40832 of the sled 40830 further comprises proximal recesses 40836, distal recesses 40839, and proximal ramps 40837. Each of the distal recesses 40839 comprises a proximal ramp 40839a and a distal wall 40839b. Further, each of the proximal recesses 40836 comprises a proximal wall 40836a tapered at angle Θₚᵣₒₓᵢₘₐₗ. In any event, the proximal ramps 40837 and the proximal recesses 40836 and are configured to permit the firing driver 40810 to selectively engage and disengage the sled 40830, as discussed in greater detail below.

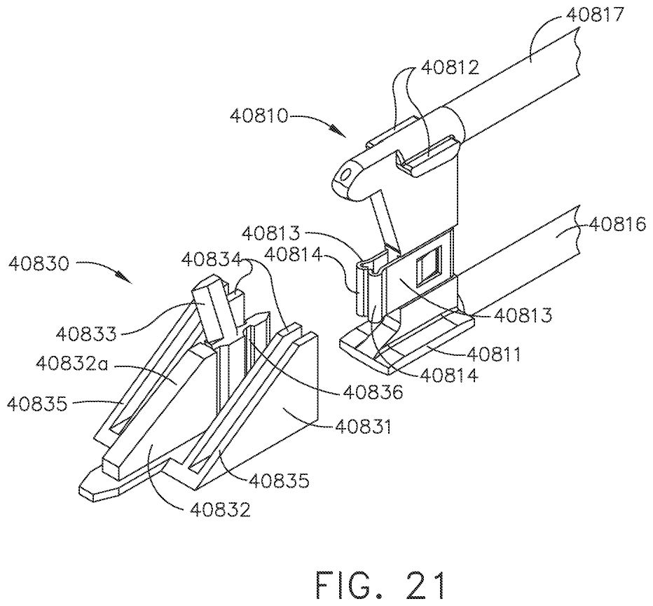

Referring primarily to FIG. 21, the firing driver 40810 comprises a first lateral cam 40811 configured to engage the first jaw of the end effector 40802 and second lateral cams 40812 configured to engage the second jaw of the end effector 40802. The first and second lateral cams 40811, 40812 can be configured to maintain the positioning of the first and second jaws of the end effector 40802 relative to one another during firing and retraction strokes of the firing driver 40810. As can be seen in FIG. 21, the firing driver 40810 further comprises a first firing bar, rod, element, driver assembly, etc. 40186 extending proximally from the firing driver 40810 and a second firing bar, rod, element, driver assembly, etc. 40817 extending proximally from the firing driver 40810. The first firing bar 40816 is spaced apart from the second firing bar 40817. The first and second firing bars 40816, 40817 are configured to transfer a firing motion from the surgical system 40800 to the firing driver 40810 to translate the firing driver 40810 distally and proximally. Further, the firing driver 40810 comprises a pair of opposing lateral spring arms 40813 extending distally from the firing driver 40810. The lateral spring arms 40813 are biased toward one another and are selectively engageable with the sled 40830 of the staple cartridge 40820, as discussed in greater detail below.

In use, the staple cartridge 40820 is inserted into the end effector 40802 with the sled 40830 in a proximal unfired position depicted in FIG. 17. The firing driver 40810 is initially distally advanced from a starting position toward the staple cartridge 40820 causing the lateral spring arms 40813 of the firing driver 40810 to engage the proximal ramps 40837 of the sled 40830 and be biased away from one another as depicted in FIG. 18. Further distal advancement of the firing driver 40810 results in hooked ends 40814 of the spring arms 40813 snapping into the proximal recesses 40836 of the nose portion 40832 of the sled 40830 to operably couple the firing driver 40810 to the sled 40830 as depicted in FIG. 18. The hooked ends 40814 of the spring arms 40813 may engage the v-cutout portion 40827 of the longitudinal slot 40824 of the staple cartridge 40820 to guide the spring arms 40813 toward the proximal recesses 40836 of the sled 40830 to aid in coupling the firing driver 40810 to the sled 40830. Further, a cartridge body feature, such as the flexible tab 40725 (see FIG. 6) may be configured to hold the sled 40830 in the proximal unfired position depicted in FIGS. 17 and 18. The sled 40830 is held stationary by the flexible tab 40725 to permit the spring arms 40813 to biased away from one another as the firing driver 40810 is advanced toward the sled 40830 to operably couple with the sled 40830. When the sled 40830 is advanced from the proximal unfired position in FIG. 17 to a distal fired position by the firing driver 40810 during the firing stroke, the biasing force of the flexible tab 40725 is overcome to permit the sled 40830 to be distally advanced beyond the proximal unfired position.

Further to the above, when the sled 40830 is in the proximal unfired position depicted in FIG. 17, the nose portion 40832 of the sled 40830 is at least partially positioned proximal to the cartridge slot protrusions 40829. Specifically, a distal ramp 40832a of the nose portion 40832 is positioned proximal to the cartridge protrusions 40829. In use, the sled 40830 is advanced by the firing driver 40810 such that the distal ramp 40832a of the nose portion 40832 of the sled 40830 engages the proximal ramps 40829a of the cartridge slot protrusions 40829 to permit the sled 40830 to pass by the cartridge slot protrusions 40829. In other words, the firing driver 40810 is configured to apply enough force to the sled 40830 to permit the sled 40830 to bypass the cartridge protrusions 40829 owing to the proximal ramps 40829a of the cartridge slot protrusions 40829.

Further to the above, the hooked ends 40814 of the spring arms 40813 are retained in their corresponding proximal recesses 40836 of the sled 40830 by the longitudinal slot 40824 as the sled 40830 is advanced and retracted through the staple cartridge 40820. In other words, the walls of the staple cartridge 40820 defining the longitudinal slot 40824 prevent the lateral spring arms 40813 from disengaging from the proximal recesses 40836 of the sled 40830 as long as the proximal recesses 40836 are positioned within the longitudinal slot 40824 with the spring arms 40813 positioned therein. Further, the hooked ends 40814 of the spring arms 40813 are configured to engage the proximal wall 40836a of the proximal recess 40836 of the sled 40830 to permit the firing driver 40810 to retract, i.e., pull, the sled 40830 proximally. As such, the firing driver 40810 is configured to advance the sled 40830 from the proximal unfired position (FIG. 17) to a distal fired position during the firing stroke to enable the wedge portion 40831 to eject the staples from the staple cartridge 40820 into patient tissue and the knife 40833 of the nose portion 40832 to incise the stapled patient tissue. Further, the firing driver 40810 is configured to retract the sled 40830 from the distal fired position to a proximal returned position (FIG. 20) during a retraction stroke owing to the engagement of the hooked ends 40814 of the spring arms 40813 with the proximal recesses 40836 of the sled 40830. As the sled 40830 approaches the proximal returned position, the distal wall 40839b of the distal recesses 40839 of the sled 40830 engage the distal faces 40829b of the cartridge slot protrusions 40829 of the staple cartridge 40820 to prevent the sled 40830 from moving further proximally from the proximal returned position in FIG. 20. Once the sled 40830 attains the proximal returned position, further proximal retraction of the firing driver 40810 causes the lateral spring arms 40813 of the firing driver 40810 to splay and disengage the proximal recesses 40836 of the sled 40830. Specifically, the proximal walls 40836a of the proximal recesses 40836 tapered at angle proximal permit the hooked ends 40814 of the spring arms 40813 to disengage from the proximal recesses 40836 of the sled 40830 and, thus, the firing driver 40810 and the sled 40830 are operably decoupled. Further, the v-cutout portion 40827 of the longitudinal slot provides space for the hooked ends 40814 of the lateral spring arms 40813 to move away from each other and out of the proximal recesses 40836 of the sled 40830.

Further to the above, when the firing driver 40810 is operably decoupled from the sled 40830 as depicted in FIG. 20, the sled 40830 remains in the proximal returned or leased positioned within the staple cartridge 40820. As such, the original proximal unfired position of the sled 40830 (FIGS. 17 and 18) is different than the return proximal position of the sled 40830 depicted in FIG. 20. Specifically, the returned or released position of the sled 40830 depicted in FIG. 20 is distal to the original proximal unfired position of the sled 40830 in FIG. 17. As such, when the staple cartridge 40820 was initially inserted into the end effector 40802 with the sled 40830 in the proximal unfired position, a firing driver lockout, such as those discussed above, is defeated to permit the firing driver 40810 to perform the firing stroke. However, when the sled 40830 is returned to the proximal returned or released position depicted in FIG. 20, the sled 40830 is distal to the original proximal unfired position and therefore can no longer defeat the firing lockout. As such, the firing driver 40810 is not permitted to perform another firing stroke when the sled 40830 is in the proximal returned position.

Various aspects of the subject matter described herein are set out in the following examples.

Example 1—A surgical staple cartridge (40720, 41700, 40820) comprising a cartridge body (40720a, 41710, 40820a) defining a proximal end portion and a distal end portion, a plurality of staple cavities (40750a, 41716, 40822) arranged longitudinally in the cartridge body, a staple driver (40750, 40823) movably supported in each staple cavity, and a sled (40722, 41720,40830) movably supported in the cartridge body. The cartridge body is configured to be operably seated in a jaw (40731) of a surgical stapling instrument (40700). Each staple driver is movable between an unfired position and a fired position. The sled is movable between an unfired position adjacent the proximal end portion to a fired position adjacent the distal end portion. When the sled is in the unfired position, the sled is configured to be releasably coupled to a firing driver (40710, 40810) of the surgical stapling instrument when the firing driver is driven distally from a starting position into coupling engagement with the sled. The cartridge body is configured to prevent the sled from decoupling from the firing driver during a firing stroke and a retraction stroke and permit the sled to decouple from the firing driver when the sled is retracted to a released position that is distal to the unfired position.

Example 2—The surgical staple cartridge of Example 1, wherein the sled (40722, 41720) further comprises a sled cavity (40726) configured to receive therein a hook portion (41717) of a movable latch arm (40718) protruding from the firing driver when the sled is in the unfired position and the firing driver is driven distally from the starting position into coupling engagement with the sled.

Example 3—The surgical staple cartridge of Example 2, wherein the cartridge body comprises a proximal ramp portion (40737) and a longitudinal cavity (40733). The proximal ramp portion (40737) is configured to guide the hook portion of the movable latch arm into hooking engagement with the sled cavity when the firing driver is driven into coupling engagement with the sled and permit the hook portion to disengage from the sled cavity when the sled has been retracted to the released position. The longitudinal cavity (40733) is configured to retain the hook portion of the movable latch arm in hooking engagement with the sled cavity during the firing stroke and the retraction stroke.

Example 4—The surgical staple cartridge of Examples 1, 2, or 3, wherein the sled is configured to defeat a firing driver lock (40705) of the surgical stapling instrument when the sled is in the unfired position, and wherein when the sled is in the released position, the sled is unable to defeat the firing driver lock.

Example 5—The surgical staple cartridge of Example 1, wherein the sled (41720) comprises a wedge portion (41721) and a nose portion (41723). The wedge portion (41721) is configured to actuate the staple drivers during the firing stroke. The nose portion (41723) is configured to be releasably coupled to the firing driver.

Example 6—The surgical staple cartridge of Example 5, wherein the nose portion is configured to releasably engage a pair of spring arms (40813) on the firing driver when the firing driver is driven distally from the starting position.

Example 7—The surgical staple cartridge of Example 6, wherein the nose portion comprises a proximal recess (40836) corresponding to each spring arm, wherein the proximal recess is configured to latchingly receive therein a portion of the corresponding spring arm when the corresponding spring arm is in a latched configuration.

Example 8—The surgical staple cartridge of Example 7, wherein the cartridge body further comprises a longitudinal slot (40824) extending from the proximal end portion to the distal end portion, wherein the longitudinal slot is sized relative to the nose portion to retain the spring arms in latching engagement with the corresponding proximal recesses during the firing stroke and the retraction stroke and permit the spring arms to move out of latching engagement with the corresponding proximal recesses when the sled is retracted to the released position.

Example 9—The surgical staple cartridge of Examples 7 or 8, wherein the nose portion further comprises a proximal ramp portion (40837) corresponding to each proximal recess and spring arm, and wherein each proximal ramp portion is configured to move the corresponding spring arm into latching engagement with the proximal recess when the firing driver is driven distally from the starting position.

Example 10—The surgical staple cartridge of Examples 1, 2, 3, 4, 5, 6, 7, 8, or 9, wherein the cartridge body further comprises at least one protrusion (40829) configured to prevent the sled from moving proximally beyond the released position during the retraction stroke.

Example 11—The surgical staple cartridge of Example 10, wherein each protrusion is configured to be retainingly received in a corresponding distal recess (40839) in the nose portion when the sled is in the released position.

Example 12—The surgical staple cartridge of Examples 1, 2, 3, 4, 5, 6, 7, 8, 9, 10, or 11, wherein the cartridge body further comprises a sled retainer tab (40725) configured to releasably retain the sled in the unfired position while permitting the sled to be distally advanced through the cartridge body by the firing driver during the firing stroke and retracted back to the released position and preventing the sled from returning to the unfired position.

Example 13—The surgical staple cartridge of Examples 1, 2, 3, 4, 5, 6, 7, 8, 9, 10, 11, or 12, wherein the sled comprises a knife (41725, 40833).

Example 14—The surgical staple cartridge of Example 13, wherein the cartridge body further comprises a proximal knife housing (40825), and wherein the knife is configured to be stored in the proximal knife housing when the sled is in the unfired position and the released position.

Example 15—The surgical staple cartridge of Examples 13 or 14, wherein the knife is movable between a deployed position in which a portion of the knife protrudes above a deck surface (41714) of the cartridge body and a retracted position in which the knife is below the deck surface.

Example 16—A surgical staple cartridge comprising a cartridge body, a plurality of staple cavities arranged longitudinally in the cartridge body, a staple driver movably supported in each staple cavity, and a sled movably supported in the cartridge body. The cartridge body defines a proximal end portion and a distal end portion. The cartridge body is configured to be operably seated in a jaw of a surgical stapling instrument. Each staple driver is movable between an unfired position and a fired position. The sled is movable between an unfired position adjacent the proximal end portion to a fired position adjacent the distal end portion. When the sled is in the unfired position, the sled is configured to be releasably coupled to a firing driver of the surgical stapling instrument when the firing driver is driven distally from a starting position into coupling engagement with the sled. The cartridge body is configured to prevent the sled from decoupling from the firing driver during a firing stroke and a retraction stroke and permit the sled to decouple from the firing driver when the sled is retracted to a released position that is distal to the unfired position.

Example 17—The surgical staple cartridge of Example 16, wherein the sled further comprises a sled cavity configured to receive therein a hook portion of a movable latch arm protruding from the firing driver when the sled is in the unfired position and the firing driver is driven distally from the starting position into coupling engagement with the sled.

Example 18—The surgical staple cartridge of Example 17, wherein the cartridge body comprises a proximal ramp portion and a longitudinal cavity. The proximal ramp portion is configured to guide the hook portion of the movable latch arm into hooking engagement with the sled cavity when the firing driver is driven into coupling engagement with the sled and permit the hook portion to disengage from the sled cavity when the sled has been retracted to the released position. The longitudinal cavity is configured to retain the hook portion of the movable latch arm in hooking engagement with the sled cavity during the firing stroke and the retraction stroke.

Example 19—The surgical staple cartridge of Examples 16, 17, or 18 wherein the sled is configured to defeat a firing driver lock of the surgical stapling instrument when the sled is in the unfired position, and wherein when the sled is in the released position, the sled is unable to defeat the firing driver lock.

Example 20—The surgical staple cartridge of Examples 16, 17, or 18, wherein the sled comprises a wedge portion and a nose portion. The wedge portion is configured to actuate the staple drivers during the firing stroke. The nose portion is configured to be releasably coupled to the firing driver.

Example 21—The surgical staple cartridge of Example 20, wherein the nose portion is configured to releasably engage a pair of spring arms on the firing driver when the firing driver is driven distally from the starting position.

Example 22—The surgical staple cartridge of Example 21, wherein the nose portion comprises a proximal recess corresponding to each spring arm, wherein the proximal recess is configured to latchingly receive therein a portion of the corresponding spring arm when the corresponding spring arm is in a latched configuration.

Example 23—The surgical staple cartridge of Example 22, wherein the cartridge body further comprises a longitudinal slot extending from the proximal end portion to the distal end portion, wherein the longitudinal slot is sized relative to the nose portion to retain the spring arms in latching engagement with the corresponding proximal recesses during the firing stroke and the retraction stroke and permit the spring arms to move out of latching engagement with the corresponding proximal recesses when the sled is retracted to the released position.

Example 24—The surgical staple cartridge of Examples 22 or 23, wherein the nose portion further comprises a proximal ramp portion corresponding to each proximal recess and spring arm, and wherein each proximal ramp portion is configured to move the corresponding spring arm into latching engagement with the proximal recess when the firing driver is driven distally from the starting position.

Example 25—The surgical staple cartridge of Examples 16, 17, 18, 19, 20, 21, 22, 23, or 24, wherein the cartridge body further comprises at least one protrusion configured to prevent the sled from moving proximally beyond the released position during the retraction stroke.

Example 26—The surgical staple cartridge of Example 25, wherein each protrusion is configured to be retainingly received in a corresponding distal recess in the nose portion when the sled is in the released position.

Example 27—The surgical staple cartridge of Examples 16, 17, 18, 19, 20, 21, 22, 23, 24, 25, 26, or 27, wherein the sled comprises a knife.

Example 28—The surgical staple cartridge of Example 27, wherein the cartridge body further comprises a proximal knife housing, and wherein the knife is configured to be stored in the proximal knife housing when the sled is in the unfired position and the released position.

Example 29—A surgical stapling instrument comprising a first jaw configured to operably support a surgical staple cartridge therein, a second jaw rotatable relative to the first jaw between an unclamped position and a clamped position, and a firing driver movable from a proximal position to a distal position during a firing stroke and movable from the distal position to the proximal position during a retraction stroke. The firing driver comprises a distal head portion and a latch arm movably coupled to the distal head portion. The latch arm comprises a hook portion configured to engage a ramp on the surgical staple cartridge operably supported in the first jaw when the firing driver is distally moved from the proximal position during the firing stroke. The ramp moves the hook portion into a cavity within a sled in the surgical staple cartridge such that when the firing driver is moved from the distal position during the retraction stroke the latch arm causes the sled to move proximally to a released position in which the hook portion disengages from the ramp and the hook portion moves out of the cavity within the sled.

Example 30—The surgical stapling instrument of Example 29, wherein the distal head portion is configured to move between a locked position wherein the distal head portion engages a lock ledge on the first jaw to prevent distal travel of the distal head portion beyond the locked position and an unlocked position wherein the distal head portion clears the lock ledge on the first jaw to move to the distal position during the firing stroke, and wherein the distal head portion is configured to be moved from the locked position to the unlocked position by the sled when the sled is in an unfired position within the surgical staple cartridge.

Example 31—The surgical stapling instrument of Examples 29 or 30, wherein the ramp is positioned to enable the hook portion to disengage from the sled at the released position which is distal to the unfired position of the sled.

Example 32—A stapling system comprising a stapling instrument and a staple cartridge. The stapling instrument comprises an end effector and a firing driver movable relative to the end effector from a proximal position to a distal position during a firing stroke and movable relative to the end effector from the distal position to the proximal position during a retraction stroke. The firing driver comprises a pair of opposing spring arms. The staple cartridge is positioned in the end effector. The staple cartridge comprises a cartridge body defining a proximal end portion and a distal end portion, a plurality of staple cavities arranged longitudinally in the cartridge body, a plurality of staples removably stored in the staple cavities, and a sled movably supported in the cartridge body. The sled is movable between an unfired position adjacent the proximal end portion to a fired position adjacent the distal end portion. When the sled is in the unfired position, the opposing spring arms of the firing driver are configured to be releasably coupled to the sled when the firing driver is driven distally from a starting position into coupling engagement with the sled. The cartridge body is configured to prevent the opposing spring arms of the firing driver from decoupling from the sled during the firing stroke and the retraction stroke and permit the opposing spring arms of the firing driver to decouple from the sled when the sled is retracted to a released position that is distal to the unfired position.

Example 33—The stapling system of Example 32, wherein the sled further comprises a sled cavity configured to receive therein a hook portion of at least one of the opposing spring arms of the firing driver when the sled is in the unfired position and the firing driver is driven distally from the starting position into coupling engagement with the sled.

Example 34—The stapling system of Examples 32 or 33, wherein the cartridge body comprises a cutout portion at its proximal end portion, wherein the cutout portion provides space for the opposing spring arms to couple with the sled during the firing stroke of the firing driver and provides space for the opposing spring arms to decouple from the sled during the retraction stroke of the firing driver.

Example 35—The stapling system of Examples 32, 33, or 34, wherein the cartridge body further comprises a sled retainer tab configured to releasably retain the sled in the unfired position while permitting the sled to be distally advanced through the cartridge body by the firing driver during the firing stroke and retracted back to the released position and preventing the sled from returning to the unfired position.

The entire disclosures of U.S. Pat. No. 11,589,865, entitled METHODS FOR CONTROLLING A POWERED SURGICAL STAPLER THAT HAS SEPARATE ROTARY CLOSURE AND FIRING SYSTEMS, which issued on Feb. 28, 2023, U.S. Pat. No. 6,978,921, entitled SURGICAL STAPLING INSTRUMENT INCORPORATING AN E-BEAM FIRING MECHANISM, which issued on Dec. 27, 2005, U.S. Pat. No. 10,213,203, entitled STAPLE CARTRIDGE ASSEMBLY WITHOUT A BOTTOM COVER, which issued on Feb. 26, 2019, U.S. Pat. No. 10,945,727, entitled STAPLE CARTRIDGE WITH DEFORMABLE DRIVER RETENTION FEATURES, which issued on Mar. 16, 2021, U.S. Pat. No. 11,234,698, entitled STAPLING SYSTEM COMPRISING A CLAMP LOCKOUT AND A FIRING LOCKOUT, which issued on Feb. 1, 2022, U.S. Pat. No. 11,540,826, entitled SURGICAL STAPLER END EFFECTOR SLED HAVING CARTRIDGE WALL SUPPORT FEATURE, which issued on Jan. 3, 2023, U.S. Pat. No. 10,299,792, entitled FASTENER CARTRIDGE COMPRISING NON-UNIFORM FASTENERS, which issued on May 28, 2019, U.S. Pat. No. 8,540,133, entitled STAPLE CARTRIDGE, which issued on Sep. 24, 2013, U.S. Pat. No. 9,788,835, entitled DEVICES AND METHODS FOR FACILITATING EJECTION OF SURGICAL FASTENERS FROM CARTRIDGES, which issued on Oct. 17, 2017, U.S. Pat. No. 10,105,142, entitled SURGICAL STAPLER WITH PLURALITY OF CUTTING ELEMENTS, which issued on Oct. 23, 2018, U.S. Pat. No. 10,537,324, entitled STEPPED STAPLE CARTRIDGE WITH ASYMMETRICAL STAPLES, which issued on Jan. 21, 2020, U.S. Pat. No. 7,669,746, entitled STAPLE CARTRIDGES FOR FORMING STAPLES HAVING DIFFERING FORMED STAPLE HEIGHTS, which issued on Mar. 2, 2010, U.S. Pat. No. 8,123,100, entitled SURGICAL STAPLING INSTRUMENTS INCLUDING A CARTRIDGE HAVING MULTIPLE STAPLE SIZES, which issued on Feb. 28, 2012, U.S. Pat. No. 7,407,075, entitled STAPLE CARTRIDGE HAVING MULTIPLE STAPLE SIZES FOR A SURGICAL STAPLING INSTRUMENT, which issued on Aug. 5, 2008, U.S. Pat. No. 10,085,749, entitled SURGICAL APPARATUS WITH CONDUCTOR STRAIN RELIEF, which issued on Oct. 2, 2018, U.S. Pat. No. 10,765,427, entitled METHOD FOR ARTICULATING A SURGICAL INSTRUMENT, which issued on Sep. 8, 2020, U.S. Pat. No. 11,291,445, entitled SURGICAL STAPLE CARTRIDGES WITH INTEGRAL AUTHENTICATION KEYS, which issued on Apr. 5, 2022, U.S. Pat. No. 8,864,007, entitled IMPLANTABLE FASTENER CARTRIDGE HAVING A NON-UNIFORM ARRANGEMENT, which issued on Oct. 21, 2014, U.S. Pat. No. 11,490,890, entitled COMPRESSIBLE NON-FIBROUS ADJUNCTS, which issued on Nov. 8, 2022, U.S. Pat. No. 10,952,724, entitled THREE DIMENSIONAL ADJUNCTS, which issued on Mar. 23, 2021, U.S. Pat. No. 9,770,245, entitled LAYER ARRANGEMENTS FOR SURGICAL STAPLE CARTRIDGES, which issued on Sep. 26, 2017, U.S. Pat. No. 10,123,798, entitled TISSUE THICKNESS COMPENSATOR COMPRISING CONTROLLED RELEASE AND EXPANSION, which issued on Nov. 13, 2018, U.S. Pat. No. 10,166,023, entitled METHOD OF APPLYING A BUTTRESS TO A SURGICAL STAPLER END EFFECTOR, which issued on Jan. 1, 2019, U.S. Pat. No. 11,207,065, entitled METHOD FOR FABRICATING SURGICAL STAPLER ANVILS, which issued on Dec. 28, 2021, U.S. Pat. No. 8,141,762, entitled SURGICAL STAPLER COMPRISING A STAPLE POCKET, which issued on Mar. 27, 2012, U.S. Pat. No. 8,876,857, entitled END EFFECTOR WITH REDUNDANT CLOSING MECHANISMS, which issued on Nov. 4, 2014, U.S. Pat. No. 9,629,631, entitled COMPOSITE DRIVE BEAM FOR SURGICAL STAPLING, which issued on Apr. 25, 2017, U.S. Patent Application Publication No. 2022/0346858, entitled METHOD FOR OPERATING A SURGICAL INSTRUMENT INCLUDING SEGMENTED ELECTRODES, which published on Nov. 3, 2022, U.S. Patent Application Publication No. 2022/0304680, entitled DRIVERS FOR FASTENER CARTRIDGE ASSEMBLIES HAVING ROTARY DRIVE SCREWS, which published on Sep. 29, 2022, U.S. Patent Application Publication No. 2022/0304679, entitled METHOD OF USING A POWERED STAPLING DEVICE, which published on Sep. 29, 2022, U.S. Patent Publication No. 2019/0298350, entitled METHODS FOR CONTROLLING A POWERED SURGICAL STAPLER THAT HAS SEPARATE ROTARY CLOSURE AND FIRING SYSTEMS, which published on Oct. 3, 2019, U.S. Patent Application Publication No. 2017/0367695, entitled STAPLE CARTRIDGE COMPRISING WIRE STAPLES AND STAMPED STAPLES, which published on Dec. 28, 2017, U.S. Patent Application Publication No. 2015/0134077, entitled SEALING MATERIALS FOR USE IN SURGICAL STAPLING, which published on May 14, 2015, U.S. Patent Application Publication No. 2018/0168615, entitled METHOD OF DEFORMING STAPLES FROM TWO DIFFERENT TYPES OF STAPLE CARTRIDGES WITH THE SAME SURGICAL STAPLING INSTRUMENT, which published on Jun. 21, 2018, U.S. Patent Application Publication No. 2018/0132849, entitled STAPLE FORMING POCKET CONFIGURATIONS FOR CIRCULAR SURGICAL STAPLER ANVIL, which published on May 17, 2018, U.S. Patent Application Publication No. 2018/0168613, entitled SURGICAL INSTRUMENTS WITH JAWS THAT ARE PIVOTABLE ABOUT A FIXED AXIS AND INCLUDE SEPARATE AND DISTINCT CLOSURE AND FIRING SYSTEMS, which published on Jun. 21, 2018, U.S. Patent Application Publication No. 2017/0319205, entitled POWERED END EFFECTOR ASSEMBLY WITH PIVOTABLE CHANNEL, which published on Nov. 9, 2017, U.S. Patent Application Publication No. 2014/0001231, entitled FIRING SYSTEM LOCKOUT ARRANGEMENTS FOR SURGICAL INSTRUMENTS, which published on Jan. 2, 2014, U.S. Patent Application Publication No. 2016/0095596, entitled APPARATUS FOR ENDOSCOPIC PROCEDURES, which published on Apr. 7, 2016, U.S. Patent Application Publication No. 2015/0297199, entitled ADAPTER ASSEMBLY WITH GIMBAL FOR INTERCONNECTING ELECTROMECHANICAL SURGICAL DEVICES AND SURGICAL LOADING UNITS, AND SURGICAL SYSTEMS THEREOF, which published on Oct. 22, 2015, U.S. Patent Application Publication No. 2022/0031351, entitled SURGICAL INSTRUMENTS WITH DIFFERENT ARTICULATION JOINT ARRANGEMENTS FOR ACCOMMODATING FLEXIBLE ACTUATORS, which published on Feb. 3, 2022, U.S. Patent Application Publication No. 2022/0031320, entitled SURGICAL INSTRUMENTS WITH FLEXIBLE FIRING MEMBER ACTUATOR CONSTRAINT ARRANGEMENTS, which published on Feb. 3, 2022, U.S. Patent Application Publication No. 2023/0119119, entitled CABLE-DRIVEN ACTUATION SYSTEM FOR ROBOTIC SURGICAL TOOL ATTACHMENT, which published on Apr. 20, 2023, International Patent Publication No. WO2018/071497, entitled STAPLER CARTRIDGE WITH AN INTEGRAL KNIFE, which published on Apr. 18, 2018, International Patent Publication No. WO2018/049211, entitled WRIST ARCHITECTURE, which published on Mar. 15, 2018, U.S. Pat. No. 11,298,129, entitled METHOD FOR PROVIDING AN AUTHENTICATION LOCKOUT IN A SURGICAL STAPLER WITH A REPLACEABLE CARTRIDGE, which issued on Apr. 12, 2022, U.S. Pat. No. 10,898,183, entitled ROBOTIC SURGICAL INSTRUMENT WITH CLOSED LOOP FEEDBACK TECHNIQUES FOR ADVANCEMENT OF CLOSURE MEMBER DURING FIRING, which issued on Jan. 26, 2021, U.S. Pat. No. 5,485,947, entitled LINEAR STAPLING MECHANISM WITH CUTTING MEANS, which issued on Jan. 23, 1996, International Patent Publication No. WO2018/049206, entitled STAPLER RELOAD DETECTION AND IDENTIFICATION, which published on Mar. 15, 2018, U.S. Patent Application Publication No. 2016/0249920, entitled Surgical fastener applying apparatus, which published on Sep. 1, 2016, U.S. Design Pat. No. D974,560, entitled STAPLE CARTRIDGE, which issued on Jan. 3, 2023, U.S. Design Pat. No. D967,421, entitled STAPLE CARTRIDGE, which issued on Oct. 18, 2022, U.S. Design Pat. No. D933,220, entitled BUTTRESS ASSEMBLY FOR A SURGICAL STAPLER, which issued on Oct. 12, 2021, U.S. Pat. No. 9,839,420, entitled TISSUE THICKNESS COMPENSATOR COMPRISING AT LEAST ONE MEDICAMENT, which issued on Dec. 12, 2017, U.S. Pat. No. 10,588,623, entitled ADHESIVE FILM LAMINATE, which issued on Mar. 17, 2020, U.S. Pat. No. 8,499,992, entitled DEVICE AND METHOD FOR CONTROLLING COMPRESSION OF TISSUE, which issued on Aug. 6, 2013, U.S.

Patent Application Publication No. 2022/0378427, entitled STAPLING INSTRUMENT COMPRISING JAW MOUNTS, which published on Dec. 1, 2022, U.S. Pat. No. 10,349,939, entitled METHOD OF APPLYING A BUTTRESS TO A SURGICAL STAPLER, which issued on Jul. 16, 2019, U.S. Pat. No. 9,386,988, entitled RETAINER ASSEMBLY INCLUDING A TISSUE THICKNESS COMPENSATOR, which issued on Jul. 12, 2016, U.S. Pat. No. 9,072,535, entitled SURGICAL STAPLING INSTRUMENTS WITH ROTATABLE STAPLE DEPLOYMENT ARRANGEMENTS, which issued on Jul. 7, 2015, and U.S. Pat. No. 9,844,369, entitled, SURGICAL END EFFECTORS WITH FIRING ELEMENT MONITORING ARRANGEMENTS, which issued on Dec. 19, 2017 are incorporated by reference herein.

The entire disclosures of:
U.S. Pat. No. 5,403,312, entitled ELECTROSURGICAL HEMOSTATIC DEVICE, which issued on Apr. 4, 1995;
U.S. Pat. No. 7,000,818, entitled SURGICAL STAPLING INSTRUMENT HAVING SEPARATE DISTINCT CLOSING AND FIRING SYSTEMS, which issued on Feb. 21, 2006;
U.S. Pat. No. 7,422,139, entitled MOTOR-DRIVEN SURGICAL CUTTING AND FASTENING INSTRUMENT WITH TACTILE POSITION FEEDBACK, which issued on Sep. 9, 2008;
U.S. Pat. No. 7,464,849, entitled ELECTRO-MECHANICAL SURGICAL INSTRUMENT WITH CLOSURE SYSTEM AND ANVIL ALIGNMENT COMPONENTS, which issued on Dec. 16, 2008;
U.S. Pat. No. 7,670,334, entitled SURGICAL INSTRUMENT HAVING AN ARTICULATING END EFFECTOR, which issued on Mar. 2, 2010;
U.S. Pat. No. 7,753,245, entitled SURGICAL STAPLING INSTRUMENTS, which issued on Jul. 13, 2010;
U.S. Pat. No. 8,393,514, entitled SELECTIVELY ORIENTABLE IMPLANTABLE FASTENER CARTRIDGE, which issued on Mar. 12, 2013;
U.S. patent application Ser. No. 11/343,803, entitled SURGICAL INSTRUMENT HAVING RECORDING CAPABILITIES, now U.S. Pat. No. 7,845,537;
U.S. patent application Ser. No. 12/031,573, entitled SURGICAL CUTTING AND FASTENING INSTRUMENT HAVING RF ELECTRODES, filed Feb. 14, 2008;
U.S. patent application Ser. No. 12/031,873, entitled END EFFECTORS FOR A SURGICAL CUTTING AND STAPLING INSTRUMENT, filed Feb. 15, 2008, now U.S. Pat. No. 7,980,443;
U.S. patent application Ser. No. 12/235,782, entitled MOTOR-DRIVEN SURGICAL CUTTING INSTRUMENT, now U.S. Pat. No. 8,210,411;
U.S. patent application Ser. No. 12/235,972, entitled MOTORIZED SURGICAL INSTRUMENT, now U.S. Pat. No. 9,050,083.
U.S. patent application Ser. No. 12/249,117, entitled POWERED SURGICAL CUTTING AND STAPLING APPARATUS WITH MANUALLY RETRACTABLE FIRING SYSTEM, now U.S. Pat. No. 8,608,045;
U.S. patent application Ser. No. 12/647,100, entitled MOTOR-DRIVEN SURGICAL CUTTING INSTRUMENT WITH ELECTRIC ACTUATOR DIRECTIONAL CONTROL ASSEMBLY, filed Dec. 24, 2009, now U.S. Pat. No. 8,220,688;
U.S. patent application Ser. No. 12/893,461, entitled STAPLE CARTRIDGE, filed Sep. 29, 2012, now U.S. Pat. No. 8,733,613;
U.S. patent application Ser. No. 13/036,647, entitled SURGICAL STAPLING INSTRUMENT, filed Feb. 28, 2011, now U.S. Pat. No. 8,561,870;
U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535;
U.S. patent application Ser. No. 13/524,049, entitled ARTICULATABLE SURGICAL INSTRUMENT COMPRISING A FIRING DRIVE, filed on Jun. 15, 2012, now U.S. Pat. No. 9,101,358;
U.S. patent application Ser. No. 13/800,025, entitled STAPLE CARTRIDGE TISSUE THICKNESS SENSOR SYSTEM, filed on Mar. 13, 2013, now U.S. Pat. No. 9,345,481;
U.S. patent application Ser. No. 13/800,067, entitled STAPLE CARTRIDGE TISSUE THICKNESS SENSOR SYSTEM, filed on Mar. 13, 2013, now U.S. Patent Application Publication No. 2014/0263552;
U.S. Patent Application Publication No. 2007/0175955, entitled SURGICAL CUTTING AND FASTENING INSTRUMENT WITH CLOSURE TRIGGER LOCKING MECHANISM, filed Jan. 31, 2006; and
U.S. Patent Application Publication No. 2010/0264194, entitled SURGICAL STAPLING INSTRUMENT WITH AN ARTICULATABLE END EFFECTOR, filed Apr. 22, 2010, now U.S. Pat. No. 8,308,040, are hereby incorporated by reference herein.

Although various devices have been described herein in connection with certain embodiments, modifications and variations to those embodiments may be implemented. Particular features, structures, or characteristics may be combined in any suitable manner in one or more embodiments. Thus, the particular features, structures, or characteristics illustrated or described in connection with one embodiment may be combined in whole or in part, with the features, structures or characteristics of one or more other embodiments without limitation. Also, where materials are disclosed for certain components, other materials may be used. Furthermore, according to various embodiments, a single component may be replaced by multiple components, and multiple components may be replaced by a single component, to perform a given function or functions. The foregoing description and following claims are intended to cover all such modification and variations.

It is worthy to note that any reference numbers included in the appended claims are used to reference exemplary embodiments/elements described in the present disclosure. Accordingly, any such reference numbers are not meant to limit the scope of the subject matter recited in the appended claims.

The devices disclosed herein can be designed to be disposed of after a single use, or they can be designed to be used multiple times. In either case, however, a device can be reconditioned for reuse after at least one use. Reconditioning can include any combination of the steps including, but not limited to, the disassembly of the device, followed by cleaning or replacement of particular pieces of the device, and subsequent reassembly of the device. In particular, a reconditioning facility and/or surgical team can disassemble a device and, after cleaning and/or replacing particular parts of the device, the device can be reassembled for subsequent use. Those skilled in the art will appreciate that reconditioning of a device can utilize a variety of techniques for disassembly, cleaning/replacement, and reassembly. Use of such techniques, and the resulting reconditioned device, are all within the scope of the present application.

The devices disclosed herein may be processed before surgery. First, a new or used instrument may be obtained and, when necessary, cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, and/or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta radiation, gamma radiation, ethylene oxide, plasma peroxide, and/or steam.

While this invention has been described as having exemplary designs, the present invention may be further modified within the spirit and scope of the disclosure. This application is therefore intended to cover any variations, uses, or adaptations of the invention using its general principles.

1-15. (canceled)

16. A surgical staple cartridge, comprising:
a cartridge body defining a proximal end portion and a distal end portion, wherein said cartridge body is configured to be operably seated in a jaw of a surgical stapling instrument;
a plurality of staple cavities arranged longitudinally in said cartridge body;
a staple driver movably supported in each said staple cavity, wherein each said staple driver is movable between an unfired position and a fired position; and
a sled movably supported in said cartridge body, wherein said sled is movable between an unfired position adjacent said proximal end portion to a fired position adjacent said distal end portion, wherein when said sled is in the unfired position, said sled is configured to be releasably coupled to a firing driver of the surgical stapling instrument when the firing driver is driven distally from a starting position into coupling engagement with said sled, and wherein said cartridge body is configured to prevent said sled from decoupling from the firing driver during a firing stroke and a retraction stroke and permit said sled to decouple from the firing driver when said sled is retracted to a released position that is distal to the unfired position.

17. The surgical staple cartridge of claim 16, wherein said sled further comprises a sled cavity configured to receive therein a hook portion of a movable latch arm protruding from the firing driver when said sled is in the unfired position and the firing driver is driven distally from the starting position into coupling engagement with said sled.

18. The surgical staple cartridge of claim 17, wherein said cartridge body comprises:
a proximal ramp portion configured to guide the hook portion of the movable latch arm into hooking engagement with said sled cavity when the firing driver is driven into coupling engagement with said sled and permit the hook portion to disengage from said sled cavity when the sled has been retracted to the released position; and
a longitudinal cavity configured to retain the hook portion of the movable latch arm in hooking engagement with said sled cavity during the firing stroke and the retraction stroke.

19. The surgical staple cartridge of claim 16, wherein said sled is configured to defeat a firing driver lock of the surgical stapling instrument when said sled is in the unfired position, and wherein when said sled is in the released position, said sled is unable to defeat the firing driver lock.

20. The surgical staple cartridge of claim 16, wherein said sled comprises:
a wedge portion configured to actuate said staple drivers during the firing stroke; and
a nose portion configured to be releasably coupled to the firing driver.

21. The surgical staple cartridge of claim 20, wherein said nose portion is configured to releasably engage a pair of spring arms on the firing driver when the firing driver is driven distally from the starting position.

22. The surgical staple cartridge of claim 21, wherein said nose portion comprises a proximal recess corresponding to each spring arm, wherein said proximal recess is configured to latchingly receive therein a portion of the corresponding spring arm when the corresponding spring arm is in a latched configuration.

23. The surgical staple cartridge of claim 22, wherein said cartridge body further comprises a longitudinal slot extending from said proximal end portion to said distal end portion, wherein said longitudinal slot is sized relative to said nose portion to retain the spring arms in latching engagement with said corresponding proximal recesses during the firing stroke and the retraction stroke and permit the spring arms to move out of latching engagement with said corresponding proximal recesses when said sled is retracted to the released position.

24. The surgical staple cartridge of claim 23, wherein said nose portion further comprises a proximal ramp portion corresponding to each said proximal recess and spring arm, and wherein each said proximal ramp portion is configured to move the corresponding spring arm into latching engagement with the proximal recess when the firing driver is driven distally from the starting position.

25. The surgical staple cartridge of claim 24, wherein said cartridge body further comprises at least one protrusion configured to prevent said sled from moving proximally beyond the released position during the retraction stroke.

26. The surgical staple cartridge of claim 25, wherein each said protrusion is configured to be retainingly received in a corresponding distal recess in said nose portion when said sled is in said released position.

27. The surgical staple cartridge of claim 16, wherein said sled comprises a knife.

28. The surgical staple cartridge of claim 27, wherein said cartridge body further comprises a proximal knife housing, and wherein said knife is configured to be stored in said proximal knife housing when said sled is in said unfired position and said released position.

29. A surgical stapling instrument, comprising:
a first jaw configured to operably support a surgical staple cartridge therein;
a second jaw rotatable relative to said first jaw between an unclamped position and a clamped position; and
a firing driver movable from a proximal position to a distal position during a firing stroke and movable from the distal position to the proximal position during a retraction stroke, wherein said firing driver comprises:
a distal head portion; and
a latch arm movably coupled to said distal head portion, wherein said latch arm comprises a hook portion configured to engage a ramp on the surgical staple cartridge operably supported in said first jaw when said firing driver is distally moved from the proximal position during the firing stroke, wherein the ramp moves said hook portion into a cavity within a sled in the surgical staple cartridge such that when said firing driver is moved from the distal position during the retraction stroke said latch arm causes said sled to move proximally to a released position in which said hook portion disengages from the ramp and said hook portion moves out of the cavity within the sled.

30. The surgical stapling instrument of claim 29, wherein said distal head portion is configured to move between a locked position wherein said distal head portion engages a lock ledge on said first jaw to prevent distal travel of said distal head portion beyond the locked position and an unlocked position wherein said distal head portion clears said lock ledge on said first jaw to move to the distal position during the firing stroke, and wherein said distal head portion is configured to be moved from the locked position to the unlocked position by the sled when the sled is in an unfired position within the surgical staple cartridge.

31. The surgical stapling instrument of claim 30, wherein the ramp is positioned to enable the hook portion to disengage from the sled at the released position which is distal to the unfired position of the sled.

32. A stapling system, comprising:
a stapling instrument, comprising:
an end effector; and
a firing driver movable relative to said end effector from a proximal position to a distal position during a firing stroke and movable relative to said end effector from said distal position to said proximal position during a retraction stroke, wherein said firing driver comprises a pair of opposing spring arms; and
a staple cartridge positioned in said end effector, wherein said staple cartridge comprises:
a cartridge body defining a proximal end portion and a distal end portion;
a plurality of staple cavities arranged longitudinally in said cartridge body;
a plurality of staples removably stored in said staple cavities; and
a sled movably supported in said cartridge body, wherein said sled is movable between an unfired position adjacent said proximal end portion to a fired position adjacent said distal end portion, wherein when said sled is in the unfired position, said opposing spring arms of said firing driver are configured to be releasably coupled to said sled when said firing driver is driven distally from a starting position into coupling engagement with said sled, and wherein said cartridge body is configured to prevent said opposing spring arms of said firing driver from decoupling from said sled during the firing stroke and the retraction stroke and permit said opposing spring arms of said firing driver to decouple from said sled when said sled is retracted to a released position that is distal to the unfired position.

33. The stapling system of claim 32, wherein said sled further comprises a sled cavity configured to receive therein a hook portion of at least one of the opposing spring arms of said firing driver when said sled is in the unfired position and said firing driver is driven distally from the starting position into coupling engagement with said sled.

34. The stapling system of claim 32, wherein said cartridge body comprises a cutout portion at its proximal end portion, wherein said cutout portion provides space for the opposing spring arms to couple with the sled during the firing stroke of the firing driver and provides space for the opposing spring arms to decouple from the sled during the retraction stroke of the firing driver.

35. The stapling system of claim 32, wherein said cartridge body further comprises a sled retainer tab configured to releasably retain said sled in the unfired position while permitting said sled to be distally advanced through said cartridge body by the firing driver during the firing stroke and retracted back to the released position and preventing said sled from returning to the unfired position.
